(19) United States
(12) Patent Application Publication
(10) Pub. No.: US 2012/0128731 A1
(43) Pub. Date: May 24, 2012

(54) BIOCOMPATIBLE CARRIER AND METHOD FOR FABRICATING THE SAME

(75) Inventor: Shu-Chen Hsieh, Kaohsiung (TW)

(73) Assignee: NATIONAL SUN YAT-SEN UNIVERSITY, Kaohsiung (TW)

(21) Appl. No.: 13/075,103

(22) Filed: Mar. 29, 2011

(30) Foreign Application Priority Data
Nov. 18, 2010 (TW) 099139619

(51) Int. Cl. A61K 47/36; A61K 31/519; A61K 31/12; A61K 47/00; A61K 47/30; A61K 47/42; A61K 31/7048; A61K 9/00; B82Y 40/00; B82Y 5/00

(52) U.S. Cl. 424/400; 514/27; 514/249; 514/678; 514/769; 514/777; 514/782; 514/774; 977/773; 977/896; 977/906

(57) ABSTRACT

The invention provides a biocompatible carrier and method for fabricating the same. The biocompatible carrier includes: a gel, and a plurality of metal nanoparticles, an organic compound or combinations thereof embedded in the gel, wherein the metal nanoparticles, the organic compound or combinations thereof are uniformly distributed in the gel.

### CROSS REFERENCE TO RELATED APPLICATIONS

This application claims priority of Taiwan Patent Application No. 099139619, filed on Nov. 18, 2010, the entirety of which is incorporated by reference herein.

### BACKGROUND OF THE INVENTION

1. Field of the Invention

The present invention relates to a biocompatible carrier and method for fabricating the same, and in particular relates to a biocompatible carrier having organic compounds and metal nanoparticles and method for fabricating the same.

2. Description of the Related Art

Because nanoparticles have several effects, such as surface effect, quantum size effect or quantum tunneling effect, they have unique electronic, physical and chemical properties. Nanoparticles are currently used in the medical industry, and it can be used as a carrier for delivery of therapeutic drugs or genes to a specific location and for releasing the drug to increase the effect of radiotherapy and chemotherapy.

In general, nanoparticles usually require a special surface coating treatment to prevent nanoparticle aggregation and make them biocompatible. A number of materials have been used as coating layers for nanoparticles, such as extran, polyvinyl alcohol (PVA), poly(ethyleneglycol) (PEG), and silicate.

However, the conventional methods for coating nanoparticles usually require tedious steps and chemicals, and the coatings are rarely effective. Thus, these methods are not conducive to being applied in mass production.

Accordingly, there is a need to develop a simple and cheap biocompatible carrier and method for fabricating the same.

### BRIEF SUMMARY OF THE INVENTION

The invention provides a method for fabricating a biocompatible carrier, comprising the following steps: (S11) providing a liquid gel aqueous solution; (S12) adding an organic compound into the liquid gel aqueous solution to form a mixed solution; and (S13) cooling the mixed solution to room temperature to form the biocompatible carrier.

The invention also provides a method for fabricating a biocompatible carrier, comprising the following steps: (S21) providing a gel; (S22) soaking the gel in a metal ion solution; (S23) removing the gel from the metal ion solution, and soaking the gel in a reducing agent; and (S24) removing the gel from the reducing reagent to obtain the biocompatible carrier, wherein a plurality of metal nanoparticles are formed in the biocompatible carrier.

The invention yet also provides a biocompatible carrier, comprising: a gel; and a plurality of metal nanoparticles, an organic compound or combinations thereof embedded in the gel, wherein the metal nanoparticles, the organic compound or combinations thereof are uniformly distributed in the gel.

A detailed description is given in the following embodiments with reference to the accompanying drawings.

### BRIEF DESCRIPTION OF THE DRAWING

For a more complete understanding of the present invention, and the advantages thereof, reference is now made to the following descriptions taken in conjunction with the accompanying drawings, in which:

FIGS. 1 to 2 show the flowchart of the method for fabricating the biocompatible carrier in accordance with the invention; and

FIG. 3 shows a cross-sectional schematic representation of the structure of the gel in accordance with the invention; and

FIG. 4 shows a cross-sectional schematic representation of the structure of the biocompatible carrier in accordance with the invention; and

FIG. 5 shows powder x-ray diffraction (XRD) patterns of the biocompatible carrier of one embodiment in accordance with the invention; and

FIG. 6 shows TEM images of the biocompatible carrier of one embodiment in accordance with the invention; and

FIG. 7 shows a hysteresis curves of the biocompatible carrier of one embodiment in accordance with the invention; and

FIG. 8 shows a cell viability assay of the biocompatible carrier of one embodiment in accordance with the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

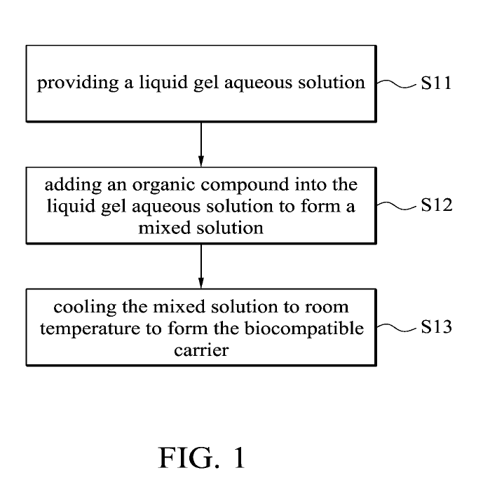

The invention provides a method for fabricating a biocompatible carrier. FIG. 1 shows a flowchart of a fabrication method of an embodiment. Firstly, in step (S11), a liquid gel aqueous solution is provided. A gel powder is dissolved in water, stirred and then heated to a temperature of 80-90° C. to form the liquid gel aqueous solution. The liquid gel aqueous solution is viscous and the gel is well dispersed in the solution. The liquid gel aqueous solution comprises hydrogel, agar, agarose, gelatin or xanthan gum. Note that the gel is not limited to above-mentioned gel, other gels that can be dissolved at high temperatures and condensed at low temperatures are also included in the scope of the invention.

In one embodiment, the agar is heated to about 80-90° C. to form the liquid gel aqueous solution.

Then, in step (S12), an organic compound is added into the liquid gel aqueous solution to form a mixed solution. The organic compound is a biocompatible molecule which has a specific property or function, such as folic acid, vitamin C, zingerone, rhodamine, rutin, phosphor material, chemical dye or combinations thereof. The phosphor material and the chemical dye can be used as a labeling agent to label the location of the biocompatible carrier. Note that in order to improve the solubility of the organic compounds, the mixed solution is stirred or other organic solvents (such as ethanol or methanol) are added into the mixed solution. Additionally, one or more organic compounds which do not react with each other may be added into the liquid gel aqueous solution, and the invention is not limited to only one organic compound.

In one embodiment, the gel powder is dissolved in water and heated to about 80-90° C. to form the liquid gel aqueous solution. Then, the solution is cooled to about 35-45° C. and the folic acid and zingerone are added into the solution.

Next, in step (S13), the mixed solution is cooled to room temperature of about 25-30° C. to form a biocompatible carrier.

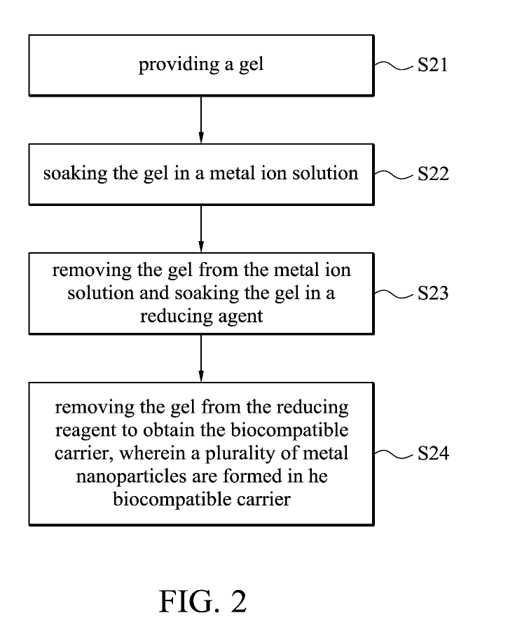

Note that the hydrogen bonds are formed between the gels due to the hydroxyl groups of the gel. The stability of the hydrogen bonds change with the temperature. The hydrogen bonds are formed at low temperature and broken at high temperature. Therefore, the liquid gel aqueous solution is formed at high temperature and a three-dimensional (3D) network-like gel (referring to FIG. 3) is formed at low temperature. The organic compounds are added into the liquid gel aqueous solution at high temperature, thus they are well distributed in the gel. After the liquid gel aqueous solution cools to room temperature, the organic compounds are thus well embedded by the gel due to the above-mentioned properties of the gel.

Additionally, after step (S12), another step may be conducted. The mixed solution may be poured into a mold, and then cooled to room temperature to form the biocompatible carrier, wherein thereafter, the biocompatible carrier is demolded from the mold. The goal of the above-mentioned steps is to define the shape of the biocompatible carrier. The shape of the biocompatible carrier depends on the shape of the mold, wherein the shape of the mold may be circular, rectangular or other shapes. Those skilled in the art may adjust the size and shape of the mold according to the actual application needs.

In one embodiment, the glass plate is used as a substrate and a circular copper ring is put on the glass plate to form a mold. Then, the mixed solution is poured into the mold to fabricate the biocompatible carrier.

Moreover, in step (S12), the metal ions and a reducing reagent are in sequence added into the mixed solution to form a plurality of the metal nanoparticles in the biocompatible carrier. The metal ions comprise magnetic metal ions, non-magnetic metal ions or combinations thereof, wherein the magnetic ions are such as iron (Fe), cobalt (Co), nickel (Ni), gadlinium (Ga), samarium (Sm), neodymium (Ne), and aluminium (Al), and non-magnetic metal ions are such as gold (Au), silver (Ag), copper (Cu), bismuth (Bi), and zinc (Zn).

The function of the reducing reagent is to conduct an oxidation-reduction reaction. The metal ions are reduced to form the metal nanoparticles. In one embodiment, a sodium hydroxide (NaOH) solution is added into a solution containing iron ions and ferrous ions (1 M Fe³⁺ and 0.5 M Fe²⁺), the then the iron ions and ferrous ions (Fe³⁺ and Fe²⁺) are precipitated and the color of the solution change from transparent to black. The precipitation reaction is described as following:

Fe²⁺+Fe³⁺+8OH⁻Fe₃O₄+4H₂O

The particle size of the metal nanoparticles are nano-sized scale and is about 5 nm-50 nm, preferably about 10 nm-40 nm and more preferably about 11 nm-30 nm.

Thus, the magnetic metal nanoparticles embedded in the biocompatible carrier may deliver the biocompatible carrier quickly and accurately to a desired location by control of the magnetic fields.

Furthermore, the invention also provides a second embodiment. FIG. 2 shows a flowchart of a fabrication method of the second embodiment. Firstly, in step (S21), a gel is provided. The formation of the gel comprises the following steps: a liquid gel aqueous solution is provided; the liquid gel aqueous solution is poured into a mold; the liquid gel aqueous solution is cooled to obtain the gel; and the gel is demolded from the mold. The gel comprises hydrogel, agar, agarose, gelatin or xanthan gum.

In one embodiment, the powder of N-isopropylacrylamide, acrylamide, N,N′-methylenebisacrylamide and ammonium persulphate ((NH₄)₂S₂O₈) are dissolved in water and methanol, then tetramethylethylenediamine is added into the mixed solution to form a hydrogel solution. Then, the hydrogel solution is poured into a mold and heated to 60° C. to form a hydrogel.

The liquid gel aqueous solution further comprises the organic compound which is as previously described, thus, is omitted here.

Then, in step (S22), the gel is soaked in a metal ion solution. The metal ion solution contains a plurality of metal ions. Note that the metal ions are adsorbed in the gel by diffusion, thus, a long soak time is needed to complete the diffusion reaction. The soak time depends on the concentration of the metal ion solution. In one embodiment, the gel is soaked in a solution containing iron ions and ferrous ions (1 MFe³⁺/0.5 MFe²⁺) for 12 hours. The metal ions comprise magnetic metal ions, non-magnetic metal ions or combinations thereof, wherein the magnetic ions are such as iron (Fe), cobalt (Co), nickel (Ni), gadlinium (Ga), samarium (Sm), neodymium (Ne), and aluminium (Al), and non-magnetic metal ions are such as gold (Au), silver (Ag), copper (Cu), bismuth (Bi), and zinc (Zn).

After step (S22) and before step (S23), a cleaning step is optionally conducted. For example, the gel is cleaned by deionized water. The purpose of the cleaning step is to remove the un-adsorbed metal ions.

Then, in step (S23), the gel is removed from the metal ion solution and the gel is soaked in a reducing agent. Thus, the metal ions are reduced to form the metal nanoparticles. In one embodiment, the reducing reagent is a solution containing hydroxyl groups, such as sodium hydroxide (NaOH), potassium hydroxide (KOH) or magnesium hydroxide (Mg(OH)₂).

In step (S24), the gel is removed from the reducing reagent to obtain the biocompatible carrier and a plurality of metal nanoparticles are formed in the biocompatible carrier.

In prior art, the metal nanoparticles are formed firstly, and then the protective coating layer is modified on the metal nanoparticles to prevent nanoparticle aggregation. However, the conventional method for coating nanoparticles usually requires tedious steps and chemicals, and the coatings are rarely effective. In the embodiment of the invention, the gel is first provided, and then the gel is soaked in the metal ion solution, thus, the metal ion is diffused into the gel. Because the three-dimensional network-like gel provides a frame, the metal ions are adsorbed onto the frame. Next, the metal ions are reduced in situ by the oxidation-reduction reaction. Thus, the aggregation of the metal nanoparticles is prevented by the help of the gel.

The biocompatible carriers of the invention are preserved by the following method. The biocompatible carriers obtained from the first embodiment or the second embodiment are washed several times to remove unwanted chemicals (such as un-adsorbed metal ions or unreacted reducing reagents). Then, the biocompatible carriers are crushed into a slurry, dried under vacuum and ground into powders. Finally, the powders of biocompatible carriers are stored under vacuum.

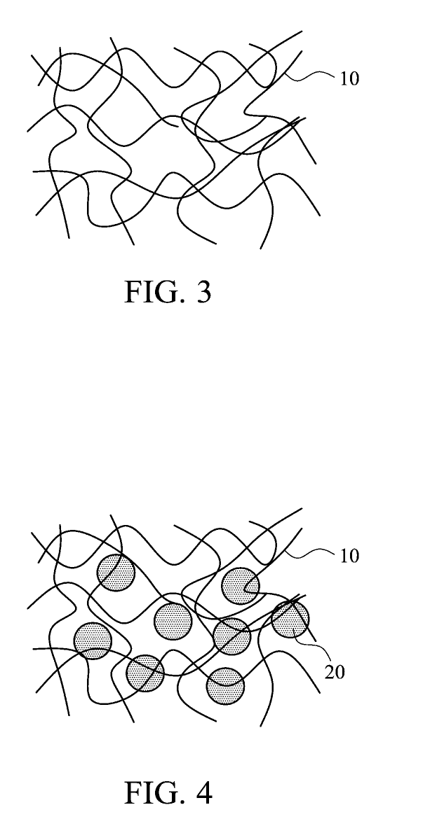

Furthermore, the invention also provides a biocompatible carrier which is fabricated by the first or second embodiments. Referring to FIG. 4, the biocompatible carrier comprises a gel 10, and a plurality of metal nanoparticles 20, the organic compound or combinations thereof which are uniformly embedded in the gel. The particle size of the metal nanoparticles is about 5-50 nm, preferably about 10-40 nm and more preferably about 11-30 nm.

The biocompatible carriers of the invention were analyzed by an x-ray diffractometer, transmission electron microscopy (TEM) or superconducting quantum interference device (SQUID). The analysis data shows that the metals were indeed embedded in the gel and thus the gel maintained a certain degree of magnetic properties. Additionally, the cell viability assay of the biocompatible carriers demonstrated that the carriers are biocompatible.

The biocompatibles carriers may also include other molecules or materials with special structures to improve the stability and function of biocompatible carriers, or even anti-cancer drugs may be added into the carrier to be used as a drug carrier.

The invention provides a simple method for fabricating an environmental friendly biocompatible carrier, and several cheap and easily available materials are used. Therefore, the biocompatible carriers are very promising for usage in various fields of drug delivery, heavy metal removal system, anti-bacterial industry, fluorescent labeling or biosensor technology.

### EXAMPLE

### Example 1-5

### Embedding of Nanoparticles or Organic Compounds in Gel

Firstly, a 5% of liquid agar gel aqueous solution was prepared and it was heated to 80-90° C. Then, the solution was cooled to about 40° C. and the composition of Example 1-5 of Table 1 were added into the solution to form a mixed solution. The mixed solution was poured into a mold (formed by a glass plate and a copper ring with a diameter of about 2 cm). The solution was cooled to room temperature to form a biocompatible carrier, and then the biocompatible carrier was demolded from the mold. In Example 1, because the gold (diameter of about 10 nm) was red, the color of the gel was changed from colorless to pale pink. In Example 2, because the FeOx was black, the color of the gel was changed from colorless to black. In Example 3-5, the color of the gels was changed from colorless to yellow.

TABLE 1

| | Example 1 | Example 2 | Example 3 | Example 4 | Example 5 |
|---|---|---|---|---|---|
| composition | 100 μl | 0.05 g | 0.05 g | 0.05 g | 0.05 g |
| | Au | FeOx | rutin | folic acid | zingerone |

### Example 6

### Embedding of Two Organic Compounds and the Nanoparticles in the Gel

Firstly, 5% of a liquid agar gel aqueous solution was prepared and it was heated to 80-90° C. Then, the solution was cooled to about 40° C. and 0.05 g of folic acid and 0.05 g of zingerone were added into the solution to form a mixed solution. The mixed solution was poured into a mold (formed by a glass plate and a copper ring with a diameter of about 2 cm). The solution was cooled to room temperature to form an agar gel, and then the agar gel was demolded from the mold. Note that one or more organic compounds which do not react with each other may be embedded in the gel.

Next, the agar gel was soaked in a solution containing iron and ferrous ions (1 M Fe³⁺/0.5 MFe²⁺) for 12 hours. Then, the agar gel was removed and washed by pure water. Then, the agar gel was soaked in sodium hydroxide solution (2.5 M NaOH, 2 ml) and it was removed from the sodium hydroxide to obtain a black agar gel.

### Example 7-12

### Embedding of Nanoparticles in the Gel

Firstly, the Example 7-12 of Table 2 of the gel was prepared. The gel was soaked in a solution containing different metal ions (see Table 2) for 12 hours. Then, the gel was removed and washed by pure water. Then, the gel was soaked in sodium hydroxide solution (2.5 M NaOH, 2 ml) and it was removed from the sodium hydroxide solution to obtain a gel with different kinds of nanoparticles.

The preparation of the hydrogel of Example 7 is described as follows:

0.2263 g of N-isopropylacrylamide, 0.1422 g of acrylamide, 0.0062 g of N,N′-methylenebisacrylamide and 0.0064 g of ammonium persulphate ((NH₄)₂S₂O₈) were dissolved in water and methanol, and then 9 μl of tetramethylethylenediamine was added into the mixed solution to form a hydrogel solution. Then, the hydrogel solution was poured into a mold and heated to 60° C. to form a hydrogel.

TABLE 2

| | Gel | Metal ions |
|---|---|---|
| Example 7 | Hydrogel | Fe³⁺/Fe²⁺ |
| Example 8 | xanthan gel | Fe³⁺/Fe²⁺ |
| Example 9 | Agarose | Fe³⁺/Fe²⁺ |
| Example 10 | Agar | Fe³⁺/Fe²⁺ |
| Example 11 | Agar | Cu²⁺ |
| Example 12 | Agar | Zn²⁺ |

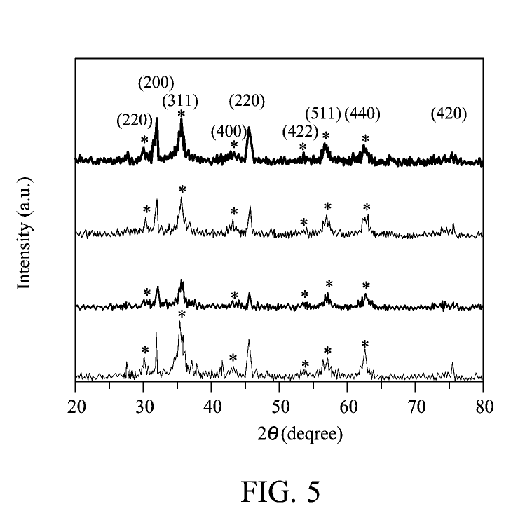

The agar gels of Example 10 with different concentrations were prepared to conduct the following analysis. The powder x-ray diffraction (XRD) patterns of Fe₃O₄ embedded in the (a) 1.25%, (b) 2.5%, (c) 5% and (d) 10% agar gels are shown in FIG. 5. The patterns indicated crystallized structures at 2θ: 30.1°, 35.4°, 43.1°, 53.4°, 57° and 62.6°, which are assigned to (220), (311), (400), (422), (511) and (440) crystallographic faces of the magnetite (reference JCPDS card No. 85-1436). Thus, the Fe₃O₄ nanoparticles were indeed embedded in the agar gels.

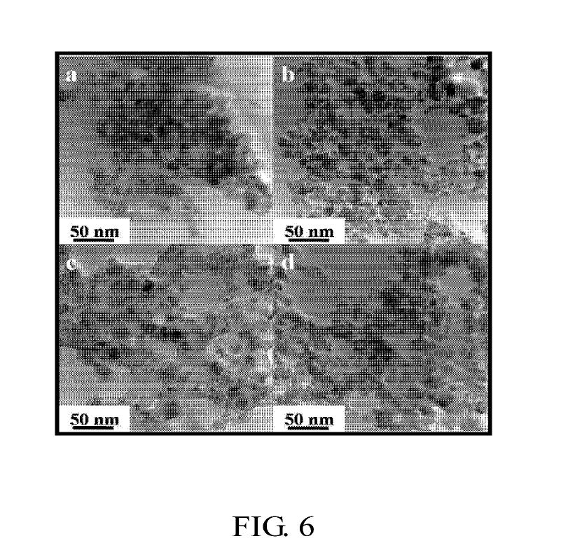

The transmission electron microscopy (TEM) images of the Fe₃O₄ embedded in the (a) 1.25%, (b) 2.5%, (c) 5% and (d) 10% agar gels are shown in FIG. 6. The Fe₃O₄ nanoparticles had particles size of about 10-30 nm and were primarily polyhedron in shape.

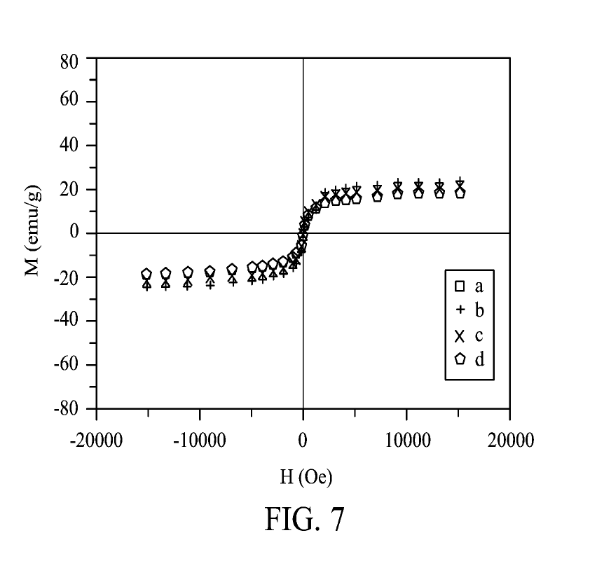

The hysteresis curves of the Fe₃O₄ embedded in the (a) 1.25%, (b) 2.5%, (c) 5% and (d) 10% agar gels are shown in FIG. 7. The saturation magnetization (Ms) of (a)-(d) was 24.3, 24.4, 20.8 and 18.7 emu/g. Thus, the method of the invention is suitable for embedding the magnetic nanoparticles, wherein even if the magnetic nanoparticles were embedded in the gel, they can still maintain a certain degree of magnetic property.

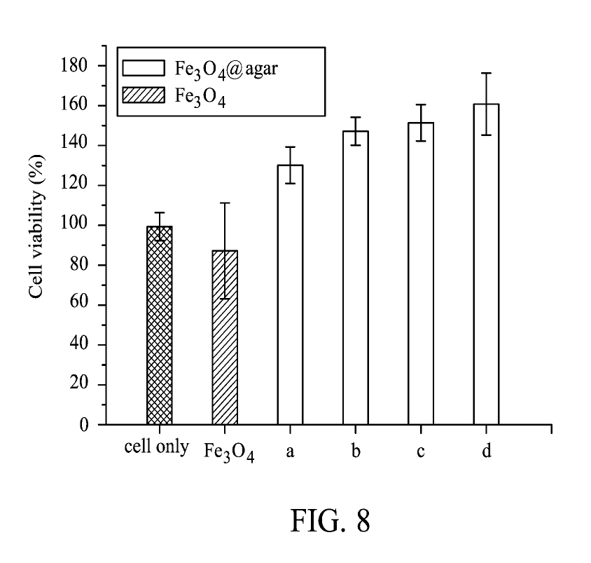

The cell viability results of the untreated cells, as well as cells treated with 500 μg/mL of bare-Fe₃O₄ and Fe₃O₄ nanoparticles embedded in the (a) 1.25%, (b) 2.5%, (c) 5% and (d) 10% agar gels are shown in FIG. 8. As shown in FIG. 8, the control test (cell only) was used as a reference, and the Fe₃O₄ nanoparticles embedded in the (a)-(d) agar gels were non-toxic to the cell. The cell viability results clearly demonstrated that the Fe₃O₄ nanoparticles that were embedded in the agar gel are biocompatible.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for fabricating a biocompatible carrier, comprising the following steps:
(S11) providing a liquid gel aqueous solution;
(S12) adding an organic compound into the liquid gel aqueous solution to form a mixed solution; and
(S13) cooling the mixed solution to room temperature to form the biocompatible carrier.

2. The method for fabricating a biocompatible carrier as claimed in claim 1, after the step (S12), further comprising:
pouring the mixed solution into a mold;
cooling the mixed solution to room temperature to form the biocompatible carrier; and
demolding the biocompatible carrier from the mold.

3. The method for fabricating a biocompatible carrier as claimed in claim 1, in the step (S12), further comprising:
adding the metal ions and a reducing reagent in sequence into the mixed solution to form a plurality of metal nanoparticles in the biocompatible carrier.

4. The method for fabricating a biocompatible carrier as claimed in claim 3, wherein the metal ions comprise iron (Fe), cobalt (Co), nickel (Ni), gadlinium (Ga), samarium (Sm), neodymium (Ne), aluminium (Al), gold (Au), silver (Ag), copper (Cu), bismuth (Bi), zinc (Zn) or combinations thereof.

5. The method for fabricating a biocompatible carrier as claimed in claim 1, wherein the liquid gel aqueous solution comprises hydrogel, agar, agarose, gelatin or xanthan gum.

6. The method for fabricating a biocompatible carrier as claimed in claim 1, wherein the organic compound comprises folic acid, vitamin C, zingerone, rhodamine, rutin, phosphor material, chemical dye or combinations thereof.

7. A method for fabricating a biocompatible carrier, comprising the following steps:
(S21) providing a gel;
(S22) soaking the gel in a metal ion solution;
(S23) removing the gel from the metal ion solution, and soaking the gel in a reducing agent; and
(S24) removing the gel from the reducing reagent to obtain the biocompatible carrier, wherein a plurality of metal nanoparticles are formed in the biocompatible carrier.

8. The method for fabricating a biocompatible carrier as claimed in claim 7, wherein the formation of the gel comprises:
providing a liquid gel aqueous solution;
pouring the liquid gel aqueous solution into a mold;
cooling the liquid gel aqueous solution to obtain the gel; and
demolding the gel from the mold.

9. The method for fabricating a biocompatible carrier as claimed in claim 8, wherein the liquid gel aqueous solution further comprises an organic compound.

10. The method for fabricating a biocompatible carrier as claimed in claim 7, wherein in the step (S22), the metal ion solution further comprises an organic compound.

11. The method for fabricating a biocompatible carrier as claimed in claim 10, wherein the organic compound comprises folic acid, vitamin C, zingerone, rhodamine, rutin, phosphor material, chemical dye or combinations thereof.

12. The method for fabricating a biocompatible carrier as claimed in claim 7, wherein the metal ion solution comprises iron (Fe), cobalt (Co), nickel (Ni), gadlinium (Ga), samarium (Sm), neodymium (Ne), aluminium (Al), gold (Au), silver (Ag), copper (Cu), bismuth (Bi), zinc (Zn) or combinations thereof.

13. The method for fabricating a biocompatible carrier as claimed in claim 7, wherein the gel comprises hydrogel, agar, agarose, gelatin or xanthan gum.

14. A biocompatible carrier, comprising:
a gel; and
a plurality of metal nanoparticles, an organic compound or combinations thereof embedded in the gel, wherein the metal nanoparticles, the organic compound or combinations thereof are uniformly distributed in the gel.

15. The biocompatible carrier as claimed in claim 14, wherein the particle size of the metal nanoparticles is about 5-50 nm.
